(19) United States
(12) Patent Application Publication
(10) Pub. No.: US 2008/0128657 A1
(43) Pub. Date: Jun. 5, 2008

(54) Biodegradable Marking Agents and Methods for Use Thereof

(76) Inventor: Tim Muldoon, West Chester, OH (US)

Correspondence Address: BROUSE MCDOWELL LPA, 388 SOUTH MAIN STREET, SUITE 500, AKRON, OH, 44311, US

(21) Appl. No.: 11/857,172

(22) Filed: Sep. 18, 2007

Related U.S. Application Data
(60) Provisional application No. 60/845,676, filed on Sep. 18, 2006.

(51) Int. Cl. G01N 33/18; G01M 3/04

(52) U.S. Cl. 252/408.1; 73/40.7

(57) ABSTRACT

A tracing agent for use in a water system having starch; water; water soluble dye; wherein the starch, water and water soluble dye are blended to form a dyed starch paste and wherein the dyed starch paste is extruded from an extruder to create a dyed starched foam.

This application claims priority to U.S. Ser. No. 60/845,676, entitled BIODEGRADABLE MARKING AGENTS AND METHODS FOR USE THEREOF, filed Sep. 18, 2006. All of the subject matter disclosed by U.S. Ser. No. 60/845,676 is hereby incorporated by reference into this application.

### I. BACKGROUND OF THE INVENTION

The present invention relates to marking and tracer agents and to the methods of use and preparations thereof.

Marking agents or tracer dyes are used for the detection of water leaks, monitoring water flow, and tracing water movement, amongst myriad other uses. Water marking agents traditionally are or contain, water soluble dyes, which are dispensed into a water system, to color the water, resulting in easier detection of the water in the system and leaks from the system. Water soluble dyes may be dispensed directly in the water system as a liquid or powder. In other applications, powdered dyes may be compacted, with other additives, including effervescing additives, to form a tablet that can be dissolved in the water system. Finally, it is known to use dyed cellulose paper as a means of conveying dye into a water system. As the paper comes into contact with the water the dye is dispersed.

Each of the existing marking agents has drawbacks. Liquid and powdered dyes can leak or spill in unwanted locations, causing undesirable staining, and these have to be stored and shipped in suitable leak proof containers. Dye tablets can break apart or crumble during transport or shipping and both dye tablets and dyed cellulose paper tend to leave a residue in the water system; namely cellulose, which can be undesirable, causing, for example, blockage in filtered water systems.

It would be desirable to provide a marking agent that does not suffer the above drawbacks. It would be desirable that the marking agent would be entirely biodegradable in the water system, without leaving any significant residue. It would be desirable to provide a marking agent that would be stable to manufacture, ship, and transport and that would readily dissolve in water to supply a suitable amount of dye into the water system. It would further be desirable to provide a marking agent that could be readily formed into a variety of shapes and sizes for use in different applications.

Starch, a readily available, known biodegradable material, has been used to prepare foamed and film products as well as other shaped products for different purposes, primarily in packaging and confectionary applications.

The use of starch in foods and confectionery products is well known. One area where starch use has been of particular interest involves expanded products such as snack foods and dry pet foods. The use of starch in the manufacture of confectionery products is disclosed in U.S. Pat. No. 3,265,509 where a mixture of high amylose starch and sugar is passed through an extruder in the presence of less than 25% moisture, to form a solid, plastic, shape-retaining confectionery mass.

While the disclosures noted above show the use of amylose containing starch materials in forming films and various other shaped products for consumption or packaging, the use of such materials as marking agents and in conjunction with water soluble dyes has not been taught.

### II. SUMMARY OF THE INVENTION

The present invention provides a biodegradable marking agent comprising a dyed, expanded starch foam, wherein the dye is a water soluble dye.

Another embodiment of this invention relates to a method of preparing biodegradable marking agents comprising blending a starch with a water soluble dye, and extruding the dyed starch mixture. The extrusion may occur within a range of suitable temperatures and other extrusion conditions.

Another embodiment of this invention relates to a method of using dyed starch foam as a marking agent, comprising forming dyed starch foam by blending a starch mixture with a water soluble dye, and extruding the dyed starch mixture, and placing an amount of the dyed starch foam in a water system. The water system may include a natural water system, such as a lake, river or stream, or a confined water system, such as a sewer system water tank, or toilet, or, in other applications, the water system may be a bathtub or pool or other container, such as may be used in the process of dying clothing or eggs.

### III. BRIEF DESCRIPTION OF THE DRAWINGS

### IV. DETAILED DESCRIPTION OF THE INVENTION

The ability to provide a marking agent which is substantially biodegradable is an important feature of this invention. The term “biodegradable” as used herein refers to the susceptibility of a substance to decomposition by living things (organisms/microorganisms) and/or natural environmental factors, e.g., the ability of compounds to be chemically broken down by bacteria, fungi, molds and yeast. It is also important, though not essential, that the marking agent be substantially or, more preferably, completely residue free. The term “residue free” as used herein refers to the state in which the marking agent dissolves without leaving any significant individual solid component in the water system, whether as a loose material or a film.

Now, in accordance with this invention, a biodegradable, substantially residue free, marking agent may be obtained by blending starch, water (as necessary) and a water soluble dye, and optionally, other additives, described below and expanding the dyed starch material through an extruder under select extrusions conditions. The expanded, dyed starch foam has excellent stability and biodegradability properties, which, coupled with its processability and formability, make it attractive for use as a water marking agent.

It is well known that starch is composed of two fractions, the molecular arrangement of one being linear and the other being branched. The linear fraction of starch is known as amylose and the branched fraction amylopectin. Starches from different sources, e.g., potato, corn, tapioca, and rice, etc., are characterized by different relative proportions of the amylose and amylopectin components. Some plant species have been genetically developed which are characterized by a large preponderance of one fraction over the other. Starch and starch blends derived from any suitable source(s), including but not limited to, corn, wheat, potatoes, and rice may be used in the present invention. In one embodiment, the starch may be derived from corn.

The starch used in this invention may be unmodified or modified and the term starch as used herein includes both types. By modified it is meant that the starch can be derivatized or modified by typical processes known in the art, e.g., esterification, etherification, oxidation, acid hydrolysis, cross-linking and enzyme conversion. Typically, modified starches include esters, such as the acetate and the half-esters of dicarboxylic acids/anhydrides, particularly the alkenylsuccinic acids/anhydrides; ethers, such as the hydroxyethyl- and hydroxypropyl starches; starches oxidized with hypochlorite; starches reacted with cross-linking agents such as phosphorus oxychloride, epichlorohydrin, hydrophobic cationic epoxides, and phosphate derivatives prepared by reaction with sodium or potassium orthophosphate or tripolyphosphate and combinations thereof.

In forming the starch mixture, it is contemplated that the starting starch material may be blended with one or more water soluble dyes. The water soluble dyes may be metered into and then blended with the starch as a liquid or powder. Suitable water soluble dyes may include water soluble natural or synthetic dyes or food colorants, including water soluble FD&C dyes, xanthene based dyes, and triphenylmethane based dyes. The water soluble dyes may also be fluorescing dyes.

Additive compounds may also be combined or blended with the starch starting material to improve physical properties or processability. Compounds such as polyvinyl alcohol, monoglyceride, and polyethylene vinyl acetate are typical additives which may be used. They are used in any amount provided the extrusion of the starch and the properties of the expanded product are suitable. Bentonite, clay or talc may also be used as all additive as described.

The method used in preparing the marking agent materials of this invention may be an extrusion process wherein the starting starch is fed into an extruder and conveyed through the apparatus under select conditions. The starch and dye may be blended in a one pass process, or a multi-pass process may be implemented, in which the starch is initially blended with water and the dye or other additives are added in a second processing step. In a preferred embodiment, the starch may be blended with water to form a starch paste, to which the dye is added and blended in the extruder. It will be noted that the starch may be blended with the dye in only suitable machine for blending, such as a mixer, prior to being extruded.

The product emerging from the extruder is an expanded, dyed starch foam. Extrusion is a conventional well known technique used in many applications for processing plastics and has been used in processing food starches to produce products such as films, foods and confectioneries and gelatinized starches.

It may be desirable to control the total moisture content of the starch material paste. By total moisture or water content is meant both the residual moisture of the starch, that is the amount pick up while stored at ambient conditions, and the amount of water fed to the extruder, individually in the dye and additives. Enough water should be present to allow the material to be processed, mixed and heated to the desired temperatures.

The apparatus used in carrying out the extrusion process may be any screw-type extruder, which may be a single or twin-screw extruder. However, any suitable extruding device may be used. Screw-type extruders will typically have rotating screws in a horizontal cylindrical barrel with an entry port mounted over one end and a shaping die mounted at the discharge end. When twin screws are used, they may be corotating and intermeshing or nonintermeshing. Each screw will comprise a helical flight or threaded section and typically will have a relatively deep feed section followed by a tapered transition section and a comparatively shallow constant-depth meter section. The screws, which are motor driven, generally fit snuggly into the cylinder or barrel to allow mixing, heating and shearing of the material as it passes through the extruder.

Control of the temperature along the length of the extruder barrel is important and is controlled in zones along the length of the screw. Heat exchange means, typically a passage, such as a channel, chamber or bore located in the barrel wall, for circulating a heated media such as oil, or an electrical heater such as calrod or coil type heaters, is often used. Additionally, heat exchange means may also be placed in or along the shaft of the screw device.

Variations in any of the elements used in the extruder may be made as desired in accordance with conventional design practices in the field.

It is also noted that the expanded starch product may be formed in different shapes by varying the size and configuration of the die opening. The product thus may be obtained in forms, such as sheets, rope or cylindrical products thereby extending the type of configuration in which it might be used. In further processing steps, the starch may be expanded into a form or sheet material may be cut to product shaped articles, such as, for example, in the shape of an animal for use by children in a bathtub, or eggs for use in dying eggs.

Having described the methods and conditions for preparation of the dyed starch foam marking agent, methods for use of these marking agents will now be described. In one preferred embodiment, the marking agent may be used as a tracer agent in the detection of water leaks, flow monitoring and tracing of water movement. For example, an amount of the dyed starch foam could be placed into a liver, lake or stream in order to detect the flow of water from the river, lake or stream. More preferably, the dyed starch foam may be used in a contained water system such as a water heater or toilet whereby, an amount of the material may be placed into the toilet or water heater and allowed to dissolve thereby releasing dye into the water. The material of the present invention has the benefit of being biodegradable in the water, without leaving a significant residue of, for example cellulose fiber, which is used in traditional marking agents. The marking agent of the present invention may also be used to detect leaks in vessels such as towers, tanks, and pipes.

It is noted that while the preferred embodiment may be used in relation to water systems, the product may be used to mark other aqueous solutions and is not expressly limited to water systems.

In another embodiment, the starch foam material, the dyed starch foam may be used for the purpose of dying a bathtub or pool to enhance the bathing or swimming experience. The use of a water soluble dye in this regard will improve clean up and will not leave residue in the drain trap or filter.

In yet another embodiment, the dyed starch foam may be used in the preparation of dyed water for dying eggs, or temporarily dying clothing.

During Easter, it is common for people to dye eggs using food coloring or other dyes dispensed in water. The marking agent of the present invention provides a useful mechanism for dispensing dye into the water and as noted previously, the marking agent may be shaped or formed into suitable strictures associated with the Easter holiday.

To further enhance the dyed starch foam marking agent of the present invention, it may be desirable to incorporate as all additive, a fragrance. In another embodiment, it may be desirable to include as an additive, an agent that will result in the production of bubbles or lather. Such an agent may include laurel sulfate. In still another embodiment, it may be desirable to include as an additive, materials that effervesce when the marking agent comes into contact with water. This may assist in causing the agent to dissolve in water.

While it is contemplated in one embodiment, that the marking agent may float on the surface of the water, on other embodiments, the density of the foam may be increased so that it will sink, or in another embodiment, the foam may be packaged in such a manner that it will sink to the bottom of the water system.

I claim:

1. A tracing agent for use in a water system comprising starch; water; water soluble dye;
wherein the starch, water and water soluble dye are blended to form a dyed starch paste and wherein the dyed starch paste is extruded from all extruder to create a dyed starched foam.

2. The tracing agent of claim 1, wherein the dye is selected from the group consisting of xanthene-based dyes, triphenylmethane dyes and F D & C dyes.

3. The tracing agent of claim 2, wherein the starch is derived from corn.

4. A method of detecting a water leak in a water system.

5. A method comprising providing a water system; providing a dyed starch foam wherein the dyed starch foam comprises starch; water; ware soluble dye; and
wherein the dyed starch foam is formed by blending the starch water and water soluble dye in an extruder to form a dyed starch paste and extruding the dyed starch paste from the extruder to form a dyed starch foam; placing an amount of dyed starch foam in the water system.

6. A method of dying water in a water system, the method comprising providing a water system having water; dissolving an amount of dyed starch foam in the water wherein the dyed starch foam comprises starch, water and water soluble dye and wherein the dyed starch foam is formed according to a process comprising the steps of blending the starch, water and dyed starch foam in a mixing apparatus to form a dyed starch paste and extruding the dyed starch paste from an extruder to form the dyed starched foam.
